(19) United States
(12) Patent Application Publication
(10) Pub. No.: US 2017/0325985 A1
(43) Pub. Date: Nov. 16, 2017

(54) Compact Elbow Brace

(71) Applicant: Christopher Johnson, Cary, NC (US)

(72) Inventor: Christopher Johnson, Cary, NC (US)

(21) Appl. No.: 15/156,276

(22) Filed: May 16, 2016

(51) Int. Cl. A61F 5/01; A63B 71/12

(52) U.S. Cl. CPC A61F 5/013; A63B 71/12; A61F 2005/0137; A61F 2005/0181

(57) ABSTRACT

The present invention provides an elbow brace which is composed of a flexible sleeve and a rigid framed brace. An elastic tubular shaped fabric sleeve is first placed over the elbow joint and has upper front and lower rear raised lips that are used to prevent the rigid and compact framed brace from slipping downwards during usage, the tight fit of the rigid brace inside the raised lip gaps of the sleeve prevents the framed brace from slipping down from the elbow joint during usage. This brace is compact in size to provide everyday comfort and reliable protection in both contact and non-contact sports.

### FIELD OF THE INVENTION

The present invention relates to elbow braces, and more particularly to a compact design elbow brace that uses a combination of both rigid and elastic components to give it unique capabilities for everyday use in both contact and non-contact sports.

### BACKGROUND OF THE INVENTION

Elbow braces in prior art generally fall into two general categories. The first common category is elbow braces made of a lightweight fabric and are usually tubular shaped to fit tightly over the elbow joint. A second common category are elbow braces that are made from rigid elements that are hinged near the pivot point of the elbow joint and are strapped directly onto the skin. Both of these prior art categories have limitations especially with athletes that want to wear braces during their normal workouts to help prevent injuries to their elbow joint.

Elbow braces that are made of lightweight fabric sleeves have the advantage of everyday usage by athletes because they are easy to put on and the lightweight fabrics of today breathe well and do not irritate the skin. However, these fabric based elbow braces do not offer a high degree of support for the joint and users of this type of elbow brace complain that they do not protect them against elbow injuries especially when participating in more demanding sports such as football and mixed martial arts. The main design flaw in these flexible braces are that they are too flexible and allow the elbow to hyperextend when put in a contact situation.

In the case of rigid fully framed elbow braces, essentially the opposite problems occur. Typical users of framed elbow braces frequently complain that they are too restrictive, large and uncomfortable for everyday use. An example would be a wide receiver attempting to outstretch a defensive player while leaping for a pass in football.

Indeed, there is a need to provide a better elbow brace that the athlete can wear everyday which addresses these noted deficiencies in the prior art.

### BRIEF SUMMARY OF THE INVENTION

It is an object of the present invention to provide an elbow brace that is lightweight and compact in size with a total length of about six inches and a total weight of roughly one pound.

It is yet another object of the present invention to provide an elbow brace that includes a slip resistant neoprene sleeve with a set of raised lips to keep the framed brace properly placed and aligned around the joint to be protected.

### BRIEF DESCRIPTION OF THE DRAWINGS

FIG. 1 is a perspective view of the compact elbow brace of the present invention.

FIG. 2 is a cross sectional view of the flexible sleeve component of the elbow brace.

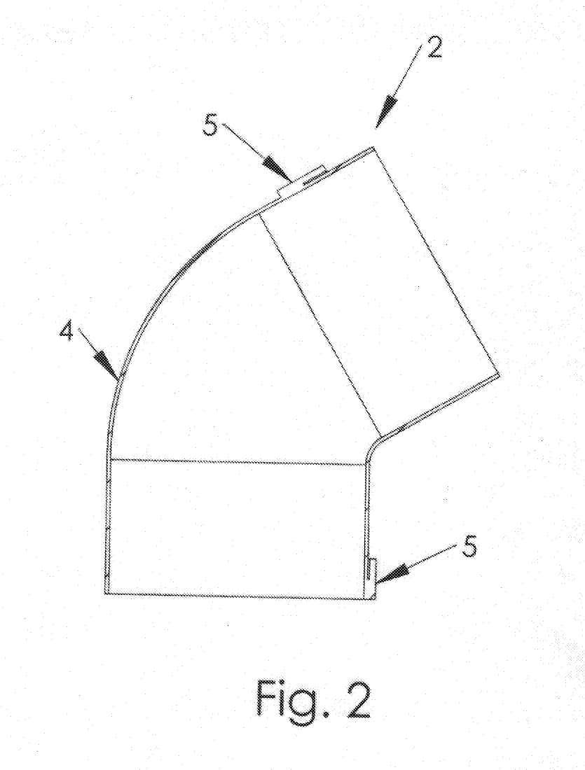

FIG. 3 is a detailed view of the upper raised lip portion of the flexible sleeve.

FIG. 4 is a perspective view of the rigid framed brace component of the elbow brace.

### DETAILED DESCRIPTION OF THE INVENTION

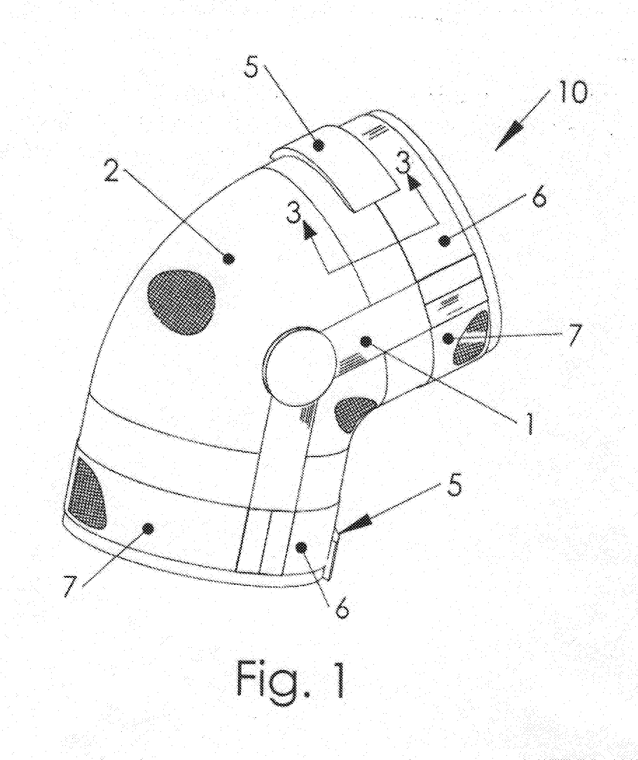

Referring now to the drawings and in particular FIG. 1, an elbow brace according to the present invention is generally designated by reference numeral 10. Elbow brace 10 consists of a compact framed brace 1 and a flexible sleeve 2. The flexible sleeve 2 in the preferred embodiment is made of a lightweight fabric that is strong and flexible, preferably a material such as neoprene rubber. The brace wearer would first slide the sleeve 2 over their hand and pull the sleeve up to fit snugly over the elbow joint. In the preferred embodiment, the sleeve 2 would be a continuous molded sleeve and not stitched together. Various sized sleeves would be molded to fit a large range of elbow joint sizes for both men and woman.

Referring next to FIGS. 1 & 2, sleeve 2 is shown to have raised lips 5 located at the front side of the sleeve for the upper raised lip and at the back side of the sleeve for the lower raised lip. In the preferred embodiment as shown in FIG. 1, the upper raised lip 5 covers only a fraction of the front half of the sleeve and the lower raised lip covers only a fraction of the rear half of the sleeve. In the preferred embodiment the raised lips 5 are molded into the main body 4 of the sleeve 2. The sleeve 2 is preferably made of an elastic material such as neoprene rubber. Neoprene rubber also has a high degree of friction which will provide the elbow brace 10 an added degree of improve slip resistance during usage as compared with typical framed braces of the prior art.

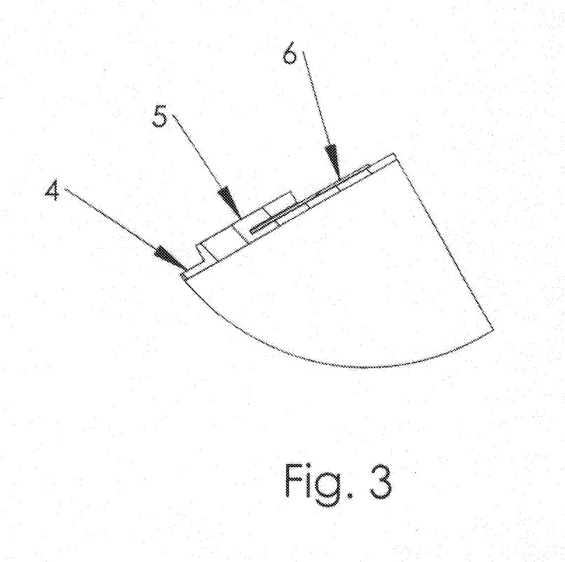

FIG. 3 shows a cross sectional view of the upper raised lip 5 and rigid arch support 6 (through line 3-3 previously drawn in FIG. 1) in order to show a close up view of how the rigid arch support 6 fits inside the raised lip 5 of sleeve 2. The raised lip creates a slight gap which allows the rigid arched support to fit inside thus forming a tight fight between the two pieces.

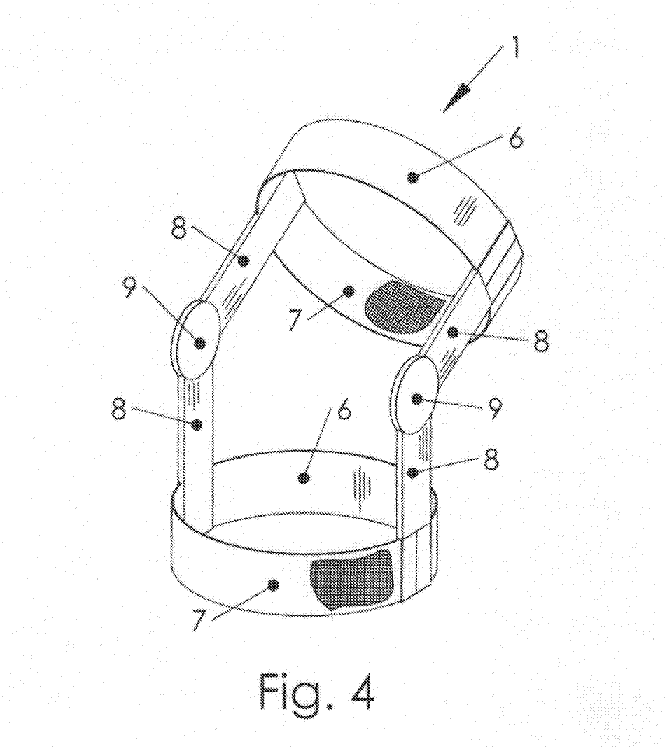

Referring next to FIG. 4, the rigid framed brace 1 is shown in perspective view to show the various components of its construction. Framed brace 1 consists of a combination of rigid and flexible support components. The rigid components are preferably made from lightweight and strong materials such as aluminum or carbon fiber composite. The flexible material in the preferred embodiment may be made from lightweight flexible materials including neoprene rubber or Velcro strap. The components that together form a rigid brace frame are the vertical support bars 8 and the upper front and lower rear rigid arched supports 6. In order to form a compact elbow brace, the length of the vertical support bars 8 is preferably between 2 to 3 inches. The center hinge 9 allows for limited angular movement of the elbow brace frame 10 during use and each hinge 9 is rigidly attached to both upper and lower vertical support bars 8 on each side of said framed brace 1.

Referring again to FIG. 4, the flexible components of rigid framed brace 1 consist of both upper rear and lower front flexible support straps 7. The flexible support straps 7 may be secured to the upper front and lower rear rigid arched supports 6 by several well known methods in the art of making elbow braces including Velcro, clips, or button snaps.

It will be obvious to those skilled in the art of making elbow braces that modifications may be made to the embodiments described above without substantially departing from the scope of the present invention. For example, an alternative embodiment of the present invention is for the wearer to only use the rigid framed brace 1 without said sleeve 2.

What is claimed is:

1. An elbow brace comprising a sleeve made of a moldable rubber compound, said sleeve having an upper raised lip portion extending directly from the front outer surface of said sleeve and a lower raised lip extending directly from the rear outer surface of said sleeve, said upper and lower lips having an open portion therein, a rigid frame member having an upper front and a lower rear rigid arch support part connected together by a pair of upper support bars and a pair of lower support bars, that are pivotally connected via a centrally located hinge component, the upper front arch frame member is located inside of the open portion of the upper raised lip portion and the lower rear arch frame member is located inside of the open portion of the lower raised lip portion, said sleeve is elongated and extends along the entire length of the frame member.

2. The elbow brace of claim 1, wherein the total combined length of the rigid brace support bars is between five to six inches in length.

3. The elbow brace of claim 1, wherein the total combined weight of the sleeve and the frame member is approximately one pound.

4. The elbow brace of claim 1, wherein a pair of upper rear and lower front flexible straps are adapted to secure the elbow to the upper front and lower rigid arch members.

5. The elbow brace of claim 1, wherein the rigid frame brace is adapted to be applied to the elbow without the use of the sleeve.
